(19) United States
(12) Patent Application Publication
(10) Pub. No.: US 2008/0234567 A1
(43) Pub. Date: Sep. 25, 2008

(54) APPARATUS AND METHOD FOR PROVIDING A NONINVASIVE DIAGNOSIS OF INTERNAL BLEEDING

(75) Inventors: Guillermo J. Tearney, Cambridge, MA (US); George Velmahos, Dover, MA (US); Brett E. Bouma, Quincy, MA (US); Benjamin J. Vakoc, Arlington, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation, Boston, MA (US)

(21) Appl. No.: 12/051,315

(22) Filed: Mar. 19, 2008

Related U.S. Application Data
(60) Provisional application No. 60/895,630, filed on Mar. 19, 2007.

(51) Int. Cl. A61B 5/00

(52) U.S. Cl. 600/407

(57) ABSTRACT

Exemplary apparatus and process can be provided for determining at least one characteristic of an anatomical structure. For example, it is possible to generate an acoustic wave in the anatomical structure using an opto-accoustic arrangement It is then possible to detect the acoustic wave and determine whether at least one blood pool is present at or in the anatomical structure as a function of at least one property of the acoustic wave. Further, it is possible to forward at least one first electro-magnetic radiation to at least one tissue of the anatomical structure, detect at least one second electro-magnetic radiation provided from the at least one tissue based on a motion of or within the at least one tissue to generate detection data, and determine the at least one characteristic of the at least one portion based on the detection data.

### CROSS-REFERENCE TO RELATED APPLICATION(S)

The present invention claims priority from U.S. Patent Application Ser. No. 60/895,630 filed on Mar. 19, 2007, the entire disclosure of which incorporated herein by reference.

### FIELD OF THE PRESENT INVENTION

The present invention relates generally apparatus and method for providing information associated with at least one portion of a sample, and in particular for providing non-invasive diagnosis of certain internal issues, including internal bleeding.

### BACKGROUND INFORMATION

Internal hemorrhage is a major cause of mortality following trauma in the civilian and military environments. The majority of trauma deaths from bleeding occur in the pre-hospital or early in-hospital phase, which is before surgical bleeding control is offered by expert physicians. For this reason, techniques are being developed to allow early post-traumatic bleeding control on the field by paramedics or even untrained personnel. These interventional methods are portable, simple, and safe. However, methods to successfully diagnose internal bleeding in the field may not exist. The developing interventional methods may be useless if the diagnosis of internal bleeding cannot be made by paramedics or untrained personnel. In this proposal, we will develop a noninvasive method for detecting hemorrhage in the abdominal cavity. Such technology may be capable of being engineered into a portable device that can be used at the point of injury and interpreted without the need of medical expertise.

Acoustooptic detection, as known in the art, utilizes a short pulse laser that irradiates a sample to generate an acoustic wave when the light interacts with an absorber internal to a body structure. In the present invention, we describe an apparatus that utilizes this effect to measure blood pools, caused by internal bleeds, inside the body. The acoustic wave generated by the optical pulse at the blood pool source, can be detected externally to the body by optical or acoustic means. The device can be portable or hand held for use in the field.

### OBJECTS AND SUMMARY OF EXEMPLARY EMBODIMENTS

Exemplary objects of the present invention may include, but not limited to the detection of blood within the internal cavities, detecting blood pools within an internal cavity by use of the acoustooptical effect, reconstructing the location and/or size of blood pool, detecting acoustic signal from within a body using optical arrangement(s), and testing the ability to identify abdominal hemorrhage in animal model of controlled bleeding:

Detection of blood within internal cavities It is one exemplary object of the present invention to provide a device for measuring the presence of internal hemorrhage via an external measurement. It is a further object of the current invention to detect the presence of blood pools within abdominal cavities. Another object of the present invention is to provide a device that can be positioned externally to a human subject to determine the presence and location of an internal bleed. It is a further object of the current invention to detect the size of a blood accumulation within an internal body cavity.

Detection of blood pools within an internal cavity by use of the acoustooptical effect. It is one of the objects of the present invention to provide a device for causing an interaction of light with blood pool that produces an acoustic signal that can be measured externally to the body. It is a further embodiment of the present invention to measure this acoustic signal generated by the blood pool by at least one of an acoustic or optical detection means. It is a further embodiment of the present invention to provide said excitation of acoustic wave and detection thereof using a portable device. It is a further embodiment of the present invention to provide said excitation of acoustic wave from said blood pool and detection using a device that is hand held.

Reconstruction of location and/or size of blood pool According to one exemplary embodiment of the present invention, it may be beneficial to a) determine appropriate pulse parameters and detection configurations by Monte Carlo and analytic modeling of excitation and phase sensitive detection in simulations designed to approximate human anatomy, b) develop an exemplary embodiment of an excitation and detection system, c) develop exemplary procedures for reconstructing internal blood sources from optically-excited acoustic data, and d) demonstrate that the exemplary embodiment of the system can identify blood pool shape and size in tissue phantoms that contain variably sized blood pools embedded in phantoms that approximate the optical properties of the body.

Detection of acoustic signal from within a body using optical arrangement(s) It is another exemplary object of the present invention to utilize an interferometric optical means to measure the acoustic wave generated internal to a body. It is a further exemplary object of the present invention to utilize at least one of low coherence interferometry, optical coherence tomography, spectral-domain optical coherence tomography, swept source optical coherence tomography, or optical frequency domain imaging, known in the art, to measuring an acoustic wave propagating in a body. It is a further object of the present invention to provide an apparatus for using aforementioned optical interferometry arrangement(s) to measure the acoustic wave emanating from a blood pool within said body.

Testing of ability to identify abdominal hemorrhage in animal model of controlled bleeding The exemplary embodiment of the device can be incorporated into a portable cart so that it can be utilized for large animal studies. Previously validated models of uncontrolled bleeding may be used, creating injuries to the spleen, liver, or inferior vena cava. These exemplary models have been repeatedly used in our lab for similar experiments. Pre- and post-injury acousto-optic excitation and optical detection measurements can be conducted at test and control sites.

Thus, exemplary embodiment of apparatus and process can be provided for determining at least one characteristic of an anatomical structure. For example, it is possible to generate an acoustic wave in the anatomical structure using an opto-accoustic arrangement It is then possible to detect the acoustic wave and determine whether at least one blood pool is present at or in the anatomical structure as a function of at least one property of the acoustic wave. Further, it is possible to forward at least one first electro-magnetic radiation to at least one tissue of the anatomical structure, detect at least one second electro-magnetic radiation provided from the at least one tissue based on a motion of or within the at least one tissue to generate detection data, and determine the at least one characteristic of the portion based on the detection data.

According to one exemplary embodiment, the portion can include a blood pool. The generation of the acoustic wave can be performed by a further arrangement which can be at least one interferometric arrangement. The interferometric arrangement may be an arrangement which receives at least one radiation from the anatomical structure and interferes the radiation with a further radiation received from a reference. The interferometric arrangement may be further configured to detect a relative phase between the radiation and the further radiation. The relative phase can be a sideband of a fundamental frequency. The further arrangement can be configured to detect the at least one characteristic as a function of the relative phase between the radiation and the further radiation.

According to still another exemplary embodiment of the present invention, the interferometric arrangement can receive the radiation from the anatomical structure and interfere the radiation with an additional radiation received from the anatomical structure. The blood pool can be provided in at least one of a Morrison's pouch, a spelenorenal space or a pelvis. For example, the opto-accoustic arrangement and the further arrangement may be provided in a hand-held device. The opto-accoustic arrangement can irradiate different portions of the anatomical structure to reconstruct a location of at least one tissue of interest. The further arrangement can generate an image of at least one portion of the tissue based on the characteristic.

These and other objects, features and advantages of the present invention will become apparent upon reading the following detailed description of embodiments of the present invention.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is an exemplary illustration of an acousto-optic effect in blood pools within an internal body cavity;

FIG. 2 is a schematic block diagram of an exemplary embodiment of an apparatus which can use optical detection technique(s) for an acoustic signal generated from a blood pool within a body cavity according to the present invention; and

FIG. 3 is a schematic block diagram of another exemplary embodiment of the apparatus using which can use the optical detection technique(s) for the acoustic signal generated from the blood pool within the body cavity according to the present invention.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention.

### DETAILED DESCRIPTION OF EXEMPLARY EMBODIMENTS

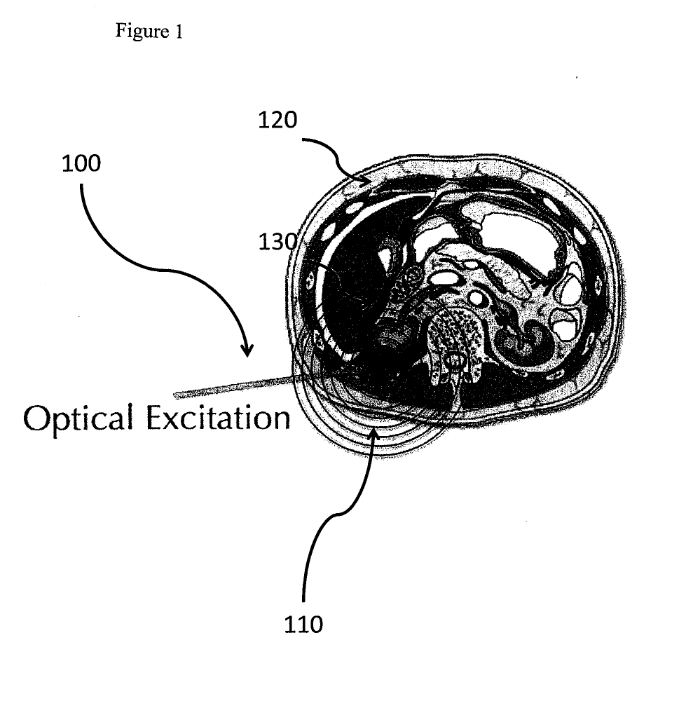

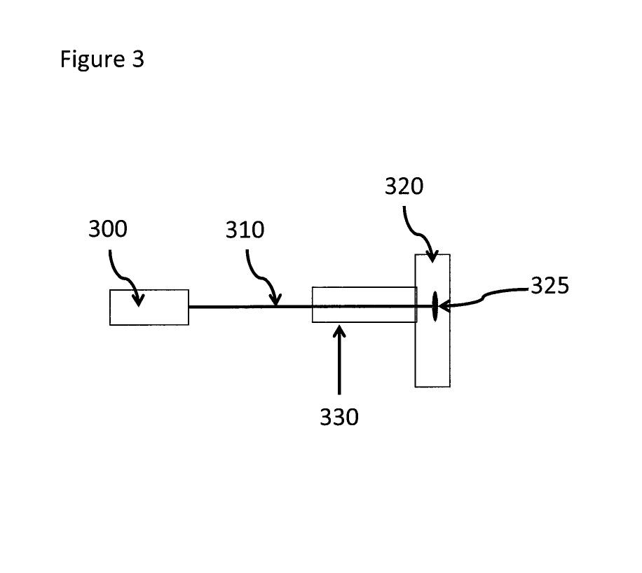

An exemplary embodiment of apparatus and method according to the present invention can utilize light 100, 210, 310 and/or other electro-magnetic radiation from for example a laser 200, 300., as shown in FIGS. 1-3. This may be done to selectively generate acoustic waves 110 emanating from blood-rich regions 130 such as pooled blood 130, 225, 325 within the body 120, 220, 320 and acoustic detection via a acoustic transducer 330 in contact with or coupled to the body 120, 220, 320 or optical interferometry to detect the resultant acoustic waves.

The acoustic waves can be generated in a manner similar to optoacoustic imaging. For example, again referring to FIGS. 1-3, a patient may be irradiated with a short pulse of light 210, 310; and the wavelength may be selected to provide differential absorption between blood and surrounding tissue and the pulse width is selected to allow stress confinement in the blood-containing region. Upon the excitation thereof, the transient pressure can rise in the blood-containing region 130, 225, 235 produces an acoustic wave 110 that propagates back to the tissue surface. Optoacoustic excitation can be more sensitive to blood than ultrasound as the generation of the acoustic waves is solely dependent on the presence of blood.

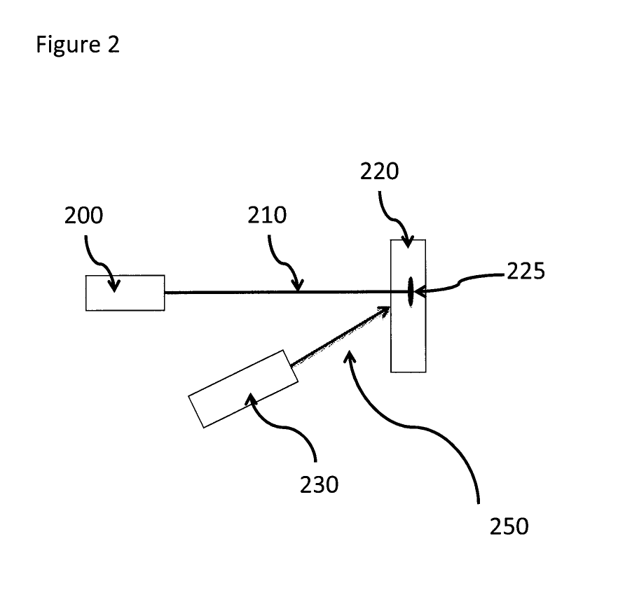

In order to detect the ultrasound waves, an optical interferometry device 230 such as low-coherence interferometry, spectral-domain OCT, and optical frequency domain imaging, as well as conventional interferometry device 230 (see FIG. 2) can be utilized to detect phase changes within the skin when it is excited by a propagating ultrasound wave 110 (see FIG. 1). As shown in FIG. 2, the tissue may be probed with long coherence, low coherence or wavelength tuned narrowband light 250. A device 230 for detecting phase sensitive low coherence ranging can detect the pressure wave or acoustic wave 110 inside or at the surface of the tissue. The interferometric measurement device 230 can have a footprint that is roughly the size of a deck of cards and a human interface the size of a pen. For example, a battery-powered, Q-switched microchip laser 200, 300 for acoustic wave excitation may not increase the form factor significantly. Alternatively, the acoustic wave can be detected by use of a conventional acoustic transducer 320.

According to one exemplary embodiment of the present invention, temporal and spatial measurements of the acoustic signal may be performed to determine, e.g., the size and shape of the blood pool distribution. The size may be determined by exciting and measuring the acoustic wave at different locations of the body 120, 220, 320 or by inputting and known optical temporal profile or frequency and measuring the temporal shape of the acoustic wave returned from the body, which is a convolution of the optical input shape and the tissue optical and acoustic response function. The exemplary knowledge, determination or estimation of this exemplary function can be used to recover the shape and/or the location of the blood pool 130.

The exemplary embodiment of the apparatus and method according to the present invention can be advantageous in that there is no requirement for any undue stabilization or surface contact, and the exemplary procedures can be performed and the exemplary apparatus may be utilized with, e.g., portable instrument(s) using a small fiber optic probe, this likely making it suitable for use by first responders. Further, the exemplary apparatus can be rapidly positioned to probe areas more likely to harbor pooled blood, including but not limited to Morrison's pouch, the spelenorenal space, and the pelvis. If the exemplary optical detection of the acoustic wave becomes untractable, it is possible, according to another exemplary embodiment of the present invention, to utilize ultrasound transducers, including piezoelectric transducers to detect the optically-generated acoustic wave.

Exemplary Optoacoustic effect in tissue Previous work conducted with optoacoustic imaging has shown that acoustic waves may be generated from foci of blood within soft tissue. For example, it has been shown that blood containing regions with diameters less than 4 mm can be detected up to 10 cm deep into tissue upon nanosecond 1064 nm pulsed irradiation. (See Esenaliev et. al, JSTQE 5:981 (1999)). The frequency response of the detected ultrasound wave according to the exemplary embodiment of the present invention can provide certain information on the size and shape of the internal acoustic source, and therefore may be utilized to discriminate blood pools from vessels or other intact, blood-rich organs.

The foregoing merely illustrates the exemplary principles of the present invention. Various modifications and alterations to the described embodiments will be apparent to those skilled in the art in view of the teachings herein. Indeed, the arrangements, systems and methods according to the exemplary embodiments of the present invention can be used with imaging systems, and for example with those described in International Patent Application PCT/US2004/029148, filed Sep. 8, 2004, U.S. patent application Ser. No. 11/266,779, filed Nov. 2, 2005, and U.S. patent application Ser. No. 10/501,276, filed Jul. 9, 2004, the disclosures of which are incorporated by reference herein in their entireties. It will thus be appreciated that those skilled in the art will be able to devise numerous systems, arrangements and methods which, although not explicitly shown or described herein, embody the principles of the present invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. An apparatus for determining at least one characteristic of an anatomical structure, comprising:
a first opto-accoustic arrangement which is configured to cause a generation of an acoustic wave in the anatomical structure; and
a second arrangement which is configured to detect the acoustic wave and determine whether at least one blood pool is present at or in the anatomical structure as a function of at least one property of the acoustic wave.

2. The apparatus according to claim 1, wherein the second arrangement is at least one interferometric arrangement.

3. The apparatus according to claim 2, wherein the interferometric arrangement is an arrangement which receives at least one radiation from the at least one anatomical structure and interferes the at least one radiation with a further radiation received from a reference.

4. The apparatus according to claim 3, wherein the interferometric arrangement is further configured to detect a relative phase between the at least one radiation and the further radiation.

5. The apparatus according to claim 4, wherein the relative phase is a sideband of a fundamental frequency.

6. The apparatus according to claim 4, wherein the second arrangement is configured to detect the at least one characteristic as a function of the relative phase between the at least one radiation and the further radiation.

7. The apparatus according to claim 2, wherein the interferometric arrangement is an arrangement which receives at least one radiation from the anatomical structure and interferes the at least one radiation with an additional radiation received from the anatomical structure.

8. The apparatus according to claim 2, wherein the blood pool is provided in at least one of a Morrison's pouch, a spelenorenal space or a pelvis.

9. The apparatus according to claim 2, wherein the first and second arrangements are provided in a hand-held device.

10. The apparatus according to claim 9, wherein the first arrangement irradiates different portions of the anatomical structure to reconstruct a location of at least one tissue of interest.

11. The apparatus according to claim 9, wherein the second arrangement generates an image of at least one portion of the tissue based on the at least one characteristic.

12. A method for determining at least one characteristic of an anatomical structure, comprising:
generating an acoustic wave in the anatomical structure in the anatomical structure using an opto-accoustic arrangement; and
detecting the acoustic wave and determining whether at least one blood pool is present at or in the anatomical structure as a function of at least one property of the acoustic wave.

13. An apparatus for determining at least one characteristic of at least one portion of an anatomical structure, comprising:
a first opto-accoustic arrangement which is configured to cause a generation of an acoustic wave in the anatomical structure; and
a second arrangement which is configured to (i) forward at least one first electro-magnetic radiation to at least one tissue of the anatomical structure, (ii) detect at least one second electro-magnetic radiation provided from the at least one tissue based on a motion of or within the at least one tissue to generate detection data, and (iii) determine the at least one characteristic of the at least one portion based on the detection data.

14. The apparatus according to claim 13, wherein the at least one portion includes a blood pool.

15. The apparatus according to claim 13, wherein the second arrangement is at least one interferometric arrangement.

16. The apparatus according to claim 15, wherein the interferometric arrangement is an arrangement which receives at least one radiation from the at least one anatomical structure and interferes the at least one radiation with a further radiation received from a reference.

17. The apparatus according to claim 16, wherein the interferometric arrangement is further configured to detect a relative phase between the at least one radiation and the further radiation.

18. The apparatus according to claim 17, wherein the relative phase is a sideband of a fundamental frequency.

19. The apparatus according to claim 17, wherein the second arrangement is configured to detect the at least one characteristic as a function of the relative phase between the at least one radiation and the further radiation.

20. The apparatus according to claim 14, wherein the interferometric arrangement is an arrangement which receives at least one radiation from the anatomical structure and interferes the at least one radiation with an additional radiation received from the anatomical structure.

21. The apparatus according to claim 14, wherein the blood pool is provided in at least one of a Morrison's pouch, a spelenorenal space or a pelvis.

22. The apparatus according to claim 14, wherein the first and second arrangements are provided in a hand-held device.

23. The apparatus according to claim 22, wherein the first arrangement irradiates different portions of the anatomical structure to reconstruct a location of at least one tissue of interest.

24. The apparatus according to claim 22, wherein the second arrangement generates an image of at least one portion of the tissue based on the at least one characteristic.

25. A method for determining at least one characteristic of at least one portion of an anatomical structure, comprising:
generating an acoustic wave in the anatomical structure using an opto-accoustic arrangement;
forwarding at least one first electro-magnetic radiation to at least one tissue of the anatomical structure;
detecting at least one second electro-magnetic radiation provided from the at least one tissue based on a motion of or within the at least one tissue to generate detection data; and
determining the at least one characteristic of the at least one portion based on the detection data.
